(19) United States
(12) Patent Application Publication
(10) Pub. No.: US 2013/0231292 A1
(43) Pub. Date: Sep. 5, 2013

(54) Synergistic anti-proliferation activity of TAS-108 with mTOR inhibitors against cancer cells

(71) Applicants: Wei Zhou, Fremont, CA (US); Lidia Sambucetti, Redwood City, CA (US)

(72) Inventors: Wei Zhou, Fremont, CA (US); Lidia Sambucetti, Redwood City, CA (US)

(21) Appl. No.: 13/784,734

(22) Filed: Mar. 4, 2013

Related U.S. Application Data
(60) Provisional application No. 61/606,130, filed on Mar. 2, 2012.

(51) Int. Cl. A61K 31/573; A61K 31/337; A61K 31/436; A61K 38/08; A61K 45/06

(52) U.S. Cl. CPC A61K 31/573; A61K 38/08; A61K 45/06; A61K 31/436; A61K 31/337
USPC 514/19.9; 514/171

(57) ABSTRACT

The invention provides methods and compositions for inhibiting tumor growth in a mammal. The methods comprise administering to the mammal a synergistic combination of (7α)-21-[4-[(Diethylamino)methyl]-2-methoxyphenoxy]-7-methyl-19-norpregna-1,3,5(10)-trien-3-ol 2-hydroxy-1,2,3-propanetricarboxylate and a synergistic chemotherapeutic drug. The combination of the compounds more than additively inhibits growth of tumor cells.

### CROSS-REFERENCE TO RELATED APPLICATION

This application claims priority to U.S. Ser. No. 61/606,130, filed Mar. 2, 2012.

### INTRODUCTION

TAS-108, a novel steroidal compound that inhibits estrogen receptors, has previously been shown to inhibit the proliferation and viability of estrogen dependent breast carcinomas in vitro and in vivo. TAS-108 also showed potent antitumor activity against tumors that are resistant to other endocrine therapies.

We studied the effects of TAS-108 with panels of targeted therapeutics on the proliferation of a variety of cancer cells, and disclose here the surprising finding of super-additive (synergistic) combination effects of TAS-108 with certain chemotherapeutic drugs.

Yamamoto, Y., et al. TAS-108, a Novel Oral Steroidal Antiestrogenic Agent, Is a Pure Antagonist on Estrogen Receptor A and a Partial Agonist on Estrogen Receptor B with Low Uterotrophic Effect. Clin. Cancer Res., 11: 315-322, 2005.

Inaji et al., Randomized phase II study of three doses of oral TAS-108 in postmenopausal patients with metastatic breast cancer. Cancer Sci. 2012 September; 103(9):1708-13. Epub 2012 Jul. 16.

### SUMMARY OF THE INVENTION

The invention provides methods and compositions for inhibiting growth of mammalian tumor cells by contacting the cells with a synergistic combination of TAS-108 and a synergistic chemotherapeutic drug, wherein the combination more than additively inhibits growth the cells. Generally, the contacting step comprises administering the combination to a mammal having a tumor comprising the cells.

In various embodiments:
one or both of the TAS-108 and drug is administered in an amount subtherapeutic if administered alone;
one or both of the TAS-108 and drug is administered in an amount less than its IC₅₀ for said cells;
the TAS-108 is administered at a dosage significantly less than 40 or at or less than 30, 20 or 10 mg;
the combination is administered in a single composition;
the drug is an mTOR inhibitor, particularly Rapamycin, Everolimus, Temsirolimus or Ridaforolimus (Deforolimus);
the drug is a taxane, particularly Paclitaxel or Docetaxel;
the drug is Actinomycin;
the cells are selected from leukemia, renal, melanoma, CNS, lung, ovarian, colon, breast, prostate, and myeloma tumor cells, particularly breast cancer cells;
the TAS-108:chemotherapeutic drug ratio is between 100:1 and 1:100, or 10:1 and 1:10;
the combination exhibits against 1, 2, 3, 4, 5 or 6 different tissue cancer cell lines selected from breast, leukemia, renal, melanoma, CNS, colon, lung, prostate, and ovarian cancer cell lines, or breast cancer cell lines selected from BT549, HS578T, MCF7, MDAMB231, MDAMB468 and T47D, greater synergy compared with a composition comprising TAS-108 and an anti-cancer agent that is Vincristine, Dasatinib, SN38, Clofarabine, FluroaraA, Imatinib, Tamoxifen, Azacytidine, Vorinostat, Nilotinib, Sunitinib, Tretinoin, Capecitabine, Lapatinib, Nelarabine, Carboplatin, Temozolomide, Gemcitabine, Celecoxib, Cytarabine, Thiotepa, Floxuridine, Cladribine or Letrozole;
the method further comprises the step of detecting a resultant inhibition of growth of the cells;

In other embodiments the invention provides a pharmaceutical composition comprising a synergistic combination, with respect to inhibiting growth of mammalian tumor cells, of TAS-108 and a synergistic chemotherapeutic drug, particularly wherein the TAS-108 and drug are coformulated in unit dosage form;

In further embodiments, the TAS-108 and/or drug are present in an amount greater than or equal to 1.0E-7M, 2.0E-7M or 3.0E-7M and/or that is less than the average IC50 for the TAS-108 and/or drug as determined in an equivalent single compound test, wherein the single compound test is against cells selected from leukemia, renal, melanoma, CNS, lung, ovarian, colon, breast, prostate, and myeloma tumor cells, particularly breast cancer cells.

The invention specifically provides all combinations of the recited aspects, as if each had been laboriously individually set forth.

### DETAILED DESCRIPTION OF PARTICULAR EMBODIMENTS

The chemical name for TAS-108 (also, SR016234) is (7α)-21-[4-[(Diethylamino)methyl]-2-methoxyphenoxy]-7-methyl-19-norpregna-1,3,5(10)-trien-3-ol 2-hydroxy-1,2,3-propanetricarboxylate, which may be structurally depicted as:

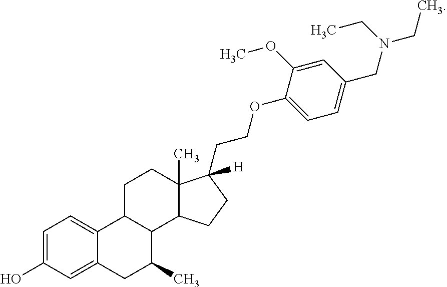 [SMILES: CCN(CC)Cc1ccc(OCC[C@@H]2CCC3C4C(CCC32C)c2ccc(O)cc2C[C@@H]4C)c(OC)c1]

TAS-108 can be present, provided or used in pharmaceutically acceptable alternative forms, such as pharmaceutically acceptable salts, esters, ethers, prodrug forms, particularly, sulfamates and phosphatese. Salts, esters, amides, pro drugs, active metabolites, analogs, and other derivatives of the active agents may be prepared using standard procedures known to those skilled in the art of synthetic organic chemistry and described, for example, by J. March, Advanced Organic Chemistry: Reactions, Mechanisms and Structure, 5th Ed. (New York: Wiley-Interscience, 2001). Furthermore, functional groups may be protected from undesired reactions during preparation or administration using protecting group chemistry; suitable protecting groups are described, for example, in Green, Protective Groups in Organic Synthesis, 3rd Ed. (New York: Wiley-Interscience, 1999).

The disclosed synergy of TAS-108 with certain chemotherapeutic drugs was surprising in that we discovered it as a result of performing a non-biased screen with many different chemotherapeutic agents representing many different mechanisms of action and only a certain few drugs demonstrated synergy at all, and very few did so across a broad panel (over 20) cancer cell lines of different tissues of origin.

In particular TAS-108 synergy was consistently found only with mTOR inhibitors, such as Rapamycin, Everolimus, Temsirolimus and Ridaforolimus (Deforolimus); taxanes, such as Paclitaxel and Docetaxel; and Actinomycin. All the other examined drugs failed to show synergy.

The synergy between TAS-108 and the synergistic chemotherapeutic drugs was unexpected, but was specific to certain drugs and classes of drugs, and could not have been predicted from any mechanistic information on this compound. The lack of synergy with all the other agents, meaning the specificity of this effect, was also surprising. Furthermore, since TAS-108 is well tolerated and does not have overlapping toxicities with the synergistic chemotherapeutic drugs the disclosed combinations are clinically relevant.

In one aspect the invention provides a method for inhibiting growth of mammalian tumor cells comprising contacting the cells with a synergistic combination of TAS-108 and a synergistic chemotherapeutic drug, wherein the combination more than additively inhibits growth the cells. By “synergism” or “synergy” is meant that the effect with the combination of agents is greater than the additive effect of the agents alone. In embodiments, synergism is determined by measuring IC₅₀ values.

In embodiments, the composition comprises the synergistic chemotherapeutic drug (and/or TAS-108) in an amount that is less than the average IC₅₀ for the drug (and/or TAS-108) as determined in an equivalent single compound test against. Such a single compound test can be conducted using the procedures described herein, or another suitable procedure. For example, the test can be against cancer cells selected from colon, ovarian, breast, prostate, and lung cancer. The conditions can be, for example, a 24-hour exposure of the cancer cells to a composition comprising the compound. For example, the combined concentration of TAS-108 and the synergistic chemotherapeutic drug is or is at least 10, 20, 30, 40, 50% less than the average IC₅₀ for TAS-108 as determined in an equivalent single compound test.

In embodiments the TAS-108 is administered at a dosage significantly less than 40 mg, preferably less than or equal to 30, 25, 20, 15, 10 or 5 mg. In embodiments, the TAS-108:drug ratio is between 100:1 and 1:100, or 10:1 and 1:10, preferably between 5:1 and 1:5, between 3:1 and 1:3, or between 2:1 and 1:2. In embodiments, more of TAS-108 is present in the composition compared with the synergistic chemotherapeutic drug (i.e., the ration is greater than 50:50). For example, the ratio of TAS-108: drug is between 90:10 and 60:40, or between 85:15 and 65:35. For example, the ratio is greater than 55:45, or 60:40, or 65:35, or 70:30, or 75:25, or 80:20. In embodiments, the amount of TAS-108 in the composition is greater than 55, 60, 65, 70, 75, 80, or 85%.

In embodiments, the composition is synergistic for at least 1, 2, 3, 4, 5 or 6 different tissue cancer cell lines selected from breast, leukemia, renal, melanoma, CNS, colon, lung, prostate, and ovarian cancer cell lines, or breast cancer cell lines selected from BT549, HS578T, MCF7, MDAMB231, MDAMB468 and T47D.

In embodiments, the composition exhibits greater synergy compared with a composition comprising TAS-108 and at least one or a plurality of anti-cancer drugs that are not mTOR inhibitors or not anti-mitotic. In embodiments, the composition exhibits greater synergy compared with a composition comprising TAS-108 and an anti-cancer drug that is Vincristine, Dasatinib, SN38, Clofarabine, FluroaraA, Imatinib, Tamoxifen, Azacytidine, Vorinostat, Nilotinib, Sunitinib, Tretinoin, Capecitabine, Lapatinib, Nelarabine, Carboplatin, Temozolomide, Gemcitabine, Celecoxib, Cytarabine, Thiotepa, Floxuridine, Cladribine or Letrozole.

In embodiments, the tumor cells to be treated are selected from ovarian, colon, breast, prostate, lung, and myeloma cancer cells, particularly present in a mammal, particularly a human. In embodiments, the growth, size or amount of the tumor or tumor cells in the mammal is reduced or suppressed, e.g. slowed by at least 20, 30, 50 or 70%. In embodiments, the tumor is a solid tumor, or a metastatic tumor.

In other aspects the invention provides:
a method for reducing the number of tumor cells in a mammal comprising: administering to the mammal a composition comprising a synergistic combination of TAS-108 and synergistic chemotherapeutic drug, wherein the administered composition is effective to substantially reduce the number of tumor cells to a level more than additive when compared to administration of TAS-108 and the drug alone;
a method for synergistically enhancing the effectiveness of a synergistic chemotherapeutic drug comprising administering an effective dose of TAS-108 in conjunction with the drug; or
a method for suppressing tumor growth in a mammal comprising: administering to the mammal a synergistic combination of TAS-108 and at least one synergistic chemotherapeutic drug, in a combined dosage effective to substantially reduce the targeted tumor cell population to a level more than additive when compared to administration of TAS-108 and the drug alone, wherein said tumor growth in said mammal is suppressed.

In an aspect, there is provided a pharmaceutical composition comprising TAS-108 and a synergistic chemotherapeutic drug wherein the pharmaceutical composition provides a synergistic effect in the treatment of a cancer selected from ovarian, colon, breast, prostate, lung, and myeloma cancer.

The composition may further comprise one or more additives such as pharmaceutically acceptable excipients, solvents, carriers, colorants, pH-modifying agents, and the like. The composition may be formulated for any appropriate mode of administration, such as oral, parenteral, transdermal, and the like. For each such mode of administration, suitable pharmaceutically acceptable additives are known in the art.

The compositions may comprise the TAS-108 and drug copackaged or coformulated together, and optionally with one or more of the additional, different anti-cancer medicaments. For example, the combinations may be coformulated, particularly in unit dosage form, or unit dosage forms of each of the TAS-108 and drug may be copackaged in a multipack adapted for sequential use, such as blisterpack, comprising sheets of unit dosage forms.

### Exemplary Coformulations:

| | Coformulated 100 mg capsules, amounts active | | |
|---|---|---|---|
| Combination | ingredients, or intravenous infusion dosages | | |
| TAS-108:Rapamycin | 40 mg:10 mg | 20 mg:5 mg | 10 mg:2 mg |
| TAS-108:Everolimus | 40 mg:10 mg | 20 mg:5 mg | 10 mg:2 mg |
| TAS-108:Temsirolimus | 40 mg:25 mg IV | 20 mg:15 mg IV | 10 mg:5 mg IV |
| TAS-108:Ridaforolimus | 40 mg:40 mg | 20 mg:20 mg | 10 mg:10 mg |
| TAS-108:Paclitaxel | 40 mg:175 mg/m² IV | 20 mg:150 mg/m² IV | 10 mg:125 mg/m² IV |
| TAS-108:Docetaxel | 40 mg:75 mg/m² IV | 20 mg:65 mg/m² IV | 10 mg:55 mg/m² IV |
| TAS-108:Actinomycin | 40 mg:12 ug/kg | 20 mg:6 ug/kg | 10 mg:3 ug/kg |

Determination of IC₅₀ values (i.e., the concentration of drug required to kill 50% of cells in exponentially growing cultures after a 24 h exposure to the drug) can be accomplished using any appropriate technique, such as the technique that follows (adapted from Liebmann et al., Br. J. Cancer (1993), 68, 1104-1109). The target cells are maintained in RPMI 1640 medium supplemented with 10% fetal bovine serum (FBS) and antibiotics, or may be grown in Dulbecco's modified minimal essential medium (DMEM) supplemented with 20% FBS and antibiotics. A number of 100 mm petri dishes are plated with 5×10⁵ cells. Exponentially growing human tumor cell lines are exposed for 24 h to drug formulated in Cremophor EL. Cells are counted after the exposure and the results plotted to determine IC₅₀.

Unless otherwise indicated, the disclosure is not limited to specific procedures, materials, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise.

The invention encompasses all combinations of recited particular and preferred embodiments. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Example 1

In this study, we examined in detail the combination effect of TAS-108 with a panel of targeted therapies on the proliferation of multiple human cancer cell lines. Cells were treated with compounds alone or in combination for three days and CellTiter-Glo® Luminescent Cell Viability Assay (Promega Corp.) was used as the endpoint to measure cell viability. Combinations of TAS-108 with mTOR inhibitors demonstrated synergistic effects on cell killing.

Cell Culture. Cells were cultured in RPMI with 10% fetal bovine serum and used between passage 2 and passage 20. Cells were harvested on Day—1 and re-suspended and plated with Wellmate (ThermoFisher) into 384 plates (BD or GBO).

Compounds. TAS-108 was synthesized in house. Other compounds used were purchased from commercial sources (Sigma, AK Scientific). 40 mM Compound stocks in 100% DMSO were made. Master plates were made with Janus (PerkinElmer), then further diluted and added to the cell plates with Matrix Platemate (Thermo) on Day 0.

Assay. CellTiter-Glo® Luminescent Cell Viability Assay (Promega Corp.) was used to measure ATP as an indicator of viable cells on Day 3 using Envision (PerkinElmer) and back up plate reader Analyst HT (MDS) and Gen5 (BioTek).

Data Analysis. Relative Luminescent Units (RLU) were plotted against corresponding drug concentrations and fitted with a standard four parameter sigmoidal curve with custom coded R program. Percentage growth inhibition is calculated as: [(Ti−Tz)/(C−Tz)]×100 for concentrations for which Ti≧Tz and [(Ti−Tz)/Tz]×100 for concentrations for which Ti<Tz. Growth inhibition of 50% (GI50) is calculated from [(Ti−Tz)/(C−Tz)]×100=50, which is the drug concentration resulting in a 50% reduction in the net increase in control cells during the drug incubation.

Table A) Concentrations of test drugs required to inhibit 50% growth of human mammary carcinoma cell lines (top table) and the maximum inhibitions with highest doses tested; NR: no effect; NQ; data didn't pass QC; HDI: Histone deacetylase Inhibitors; SERM: Selective estrogen receptor modulators.

Table C) Concentration of test drugs used in the combination screen. Five doses of TAS-108 were combined with four doses of a second compound and with DMSO control. Twenty-five doses were tested for each pair of combinations.

Table D) Observation of synergistic effect of TAS 108 paired with 9 different compounds. N: no synergistic effect; Y: synergistic effect; y: weak synergistic effect. Synergy was confirmed with dose response curves from the combo screen.

Summary of TAS-108 Activities:

1. The affinities of TAS-108 for binding ERalpha or ERbeta are similar to those of 4-OH-Tamoxifen and 17-beta-estradiol (E2)¹.

2. TAS-108 has potent anti-tumor activity against Tamoxifen-sensitive and Tamoxifen-resistant breast carcinomas in vitro and in vivo^{1.3}. TAS-108 also inhibits the growth of breast carcinoma cells with aromatose inhibitor resistant characteristics³.

3. TAS-108 has significantly less uterotrophic activity compared to Tamoxifen^{1.2}.

4. TAS-108 exhibits unique intranuclear events in tumor, uterine and other normal tissues⁴.

5. TAS-108 protects against reductions in bone density, similar to Tamoxifen².

6. In a Phase II clinical trial TAS-108 provided clinical benefits and was well tolerated².

7. TAS-108 inhibited the growth of cancer cells synergistically with mTOR inhibitors.

### REFERENCES

1. Yamamoto, Y., et al. TAS-108, a Novel Oral Steroidal Antiestrogenic Agent, Is a Pure Antagonist on Estrogen Receptor A and a Partial Agonist on Estrogen Receptor B with Low Uterotrophic Effect. Clin. Cancer Res., 11: 315-322, 2005.

2. Buzdar, M., et al. Randomized Double-Blind Phase 2 Trial of Doses of TAS-108 in Patients with Advanced or Metastatic Postmenopausal Breast Cancer, Cancer, Advanced publication November 2011.

3. Yamamoto, Y., et al. Keystone Symposia, The potency of the novel antiestrogen TAS-108 as an antagonist against tamoxifen-resistant tumors and a tissue-selective agonist. Nuclear Receptor Superfamily, D4, 2002.

4. Yamamoto, Y., et al, Both N- and C-terminal transactivation functions of DNA-bound ERalpha are blocked by a novel synthetic estrogen ligand. BBRC, 312: 656-662, 2003.

TABLE A

| | Tyrosine Kinase Inhibitors | | | | | | |
|---|---|---|---|---|---|---|---|
| | Dasatinib | Erlotinib | Imatinib | Lapatinib | Nilotinib | Sorafenib | Sunitinib |
| GI50 | | | | | | | |
| BT549 | 5.8E−08 | NR | 2.9E−05 | NR | NR | NR | 4.2E−06 |
| HS578T | 1.5E−08 | NR | NR | NR | NR | NR | 6.9E−06 |
| MCF7 | NR | NR | NR | NR | NR | NR | NR |
| MDAMB231 | 9.9E−09 | NR | 2.6E−05 | 1.4E−05 | 1.4E−05 | NR | 5.6E−06 |
| MDAMB468 | 1.6E−05 | 7.6E−05 | NQ | 1.9E−06 | NR | NR | 2.1E−05 |
| T47D | 8.6E−07 | NQ | NR | 5.8E−06 | NR | NR | NR |
| Max | | | | | | | |
| Inhibition | | | | | | | |
| BT549 | 0.51 | NR | NR | NR | NR | NR | 1.00 |
| HS578T | 0.79 | NR | NR | NR | NR | NR | 0.65 |
| MCF7 | NR | NR | NR | NR | NR | NR | NR |
| MDAMB231 | 0.82 | NR | 0.91 | 0.98 | NR | NR | 0.99 |
| MDAMB468 | 0.40 | 0.34 | NQ | 0.99 | NR | NR | NR |
| T47D | 0.37 | NQ | NR | 0.31 | NR | NR | NR |

| | HDI | mTOR inhibitors | | SERM | | |
|---|---|---|---|---|---|---|
| | Romidepsin | Everolimus | Rapamycin | TAS108 | Raloxifene | Faslodex |
| GI50 | | | | | | |
| BT549 | NQ | 5.8E−06 | 2.2E−07 | 1.3E−05 | NR | NR |
| HS578T | 3.0E−10 | 6.3E−08 | 1.0E−06 | 8.2E−06 | NR | NR |
| MCF7 | 4.4E−10 | 1.7E−07 | 5.4E−08 | 1.0E−05 | 2.5E−05 | 1.0E−06 |
| MDAMB231 | NQ | 5.4E−05 | 1.1E−05 | 9.3E−06 | NR | NR |
| MDAMB468 | NQ | 6.3E−08 | 6.3E−08 | 1.8E−06 | NR | NR |
| T47D | 1.1E−09 | 5.2E−08 | 4.7E−08 | 1.1E−05 | NQ | NR |
| Max | | | | | | |
| Inhibition | | | | | | |
| BT549 | 0.998 | 1.00 | 0.99 | 1.00 | NR | NR |
| HS578T | 0.996 | 0.41 | 0.41 | 1.00 | NR | NR |
| MCF7 | 0.94 | 0.43 | 0.55 | 0.99 | 0.27 | 0.30 |
| MDAMB231 | 0.999 | 0.40 | 0.95 | 1.00 | NR | NR |
| MDAMB468 | 0.998 | 1.00 | 1.00 | 1.00 | NR | NR |
| T47D | 0.967 | 0.49 | 0.45 | 1.00 | NQ | 0.24 |

TABLE C

| Drug (M) | D1 | D2 | D3 | D4 | D5 |
|---|---|---|---|---|---|
| TAS-108 | 2.0E−05 | 2.0E−06 | 2.0E−07 | 2.0E−08 | 2.0E−09 |
| Dasatinib | 1.0E−07 | 1.0E−08 | 1.0E−09 | 1.0E−10 | NA |
| Erlotinib | 2.0E−06 | 2.0E−07 | 2.0E−08 | 2.0E−09 | NA |
| Imatinib | 5.0E−06 | 5.0E−07 | 5.0E−08 | 5.0E−09 | NA |
| Lapatinib | 3.0E−06 | 3.0E−07 | 3.0E−08 | 3.0E−09 | NA |
| Nilotinib | 1.0E−06 | 1.0E−07 | 1.0E−08 | 1.0E−09 | NA |
| Sorafenib | 2.0E−05 | 2.0E−06 | 2.0E−07 | 2.0E−08 | NA |
| Sunitinib | 1.0E−06 | 1.0E−07 | 1.0E−08 | 1.0E−09 | NA |
| Romidepsin | 1.0E−08 | 1.0E−09 | 1.0E−10 | 1.0E−11 | NA |
| Everolimus | 1.0E−07 | 1.0E−08 | 1.0E−09 | 1.0E−10 | NA |
| Rapamycin | 1.0E−07 | 1.0E−08 | 1.0E−09 | 1.0E−10 | NA |

TABLE D

| | Tyrosine Kinase Inhibitors | | | | | | | HDI | mTOR inhibitors | |
|---|---|---|---|---|---|---|---|---|---|---|
| Cell Type | Dasatinib | Erlotinib | Imatinib | Lapatinib | Nilotinib | Sorafenib | Sunitinib | Romidepsin | Everolimus | Rapamycin |
| BT-549 | N | N | N | N | N | N | N | N | y | y |
| HS 578T | y | N | N | N | N | N | N | N | Y | Y |
| MCF7 | N | N | N | N | N | N | N | N | y | Y |
| MDA-MB- | y | N | N | N | N | N | N | N | y | y |
| 231 | | | | | | | | | | |
| MDA-MB- | N | N | N | N | N | N | N | N | y | y |
| 468 | | | | | | | | | | |
| T-47D | N | N | N | N | N | N | N | N | y | y |
| Synergy # | 2 | 0 | 0 | 0 | 0 | 0 | 0 | 0 | 6 | 6 |

### Example 2

We performed a non-bias screen of TAS108 in combination with 30 different standard chemotherapeutic agents representing many different mechanisms of action, including Actinomycin, Azacytidine, Capecitabine, Carboplatin, Celecoxib, Cladribine, Clofarabine, Cytarabine, Dasatinib, Docetaxel, Everolimus, Floxuridine, FluroaraA, Gemcitabine, Imatinib, Lapatinib, Letrozole, Nelarabine, Nilotinib, Paclitaxel, Rapamycin, Romidepsin, SN38, Sunitinib, Tamoxifen, Temozolomide, Thiotepa, Tretinoin, Vincristine and Vorinostat. We used 5×4 factorial (checkerboard) design. The combinations were screened against 60 cancer cell lines of different tissues of origin, including melanoma, ovarian, CNS, renal, lung, breast, colon, prostate, and leukemia cancers, represented by cell lines including LOXIMVI, M14,MDAMB468, HT29, NCIH522, HS578T, RPMI8226, HOP92, HL60, OVCAR8, SR, SKMEL2, PC3, HOP62, HCC2998,786O, U251, OVCAR3, NCIH23, T47D, SF295, MOLT4, HCT15, SF539, BT549, SN12C, KM12, ADRRES, A498, NCIH226, NCIH322M, OVCAR5, MALME, OVCAR4, RXF393, SW620, CCRFCEM, UACC62, ACHN, SKOV3, SF268, EKVX, A549, NCIH460, IGROV1, SNB75, UACC257, UO31, HCT116, TK10, K562, SKMEL28, COLO205, SNB19, MDAMB231, DU145, CAKI1, MDAMB435, and MCF7.

We developed a statistical method to quantify the degree of synergy beyond simple additive activity of two compounds and to calculate the standard error and statistical significance of the observed synergy. After quantifying the degree of synergy for specified doses of compounds within each individual cell line, we used meta analysis to aggregate those effects across doses and the cell line panel (Table 1).

TABLE 1
Combination of TAS-108 with 30 chemotherapeutic agents.

| Compound | ES^{A} | seES^{B} | p^{C} | Count^{D} |
|---|---|---|---|---|
| Docetaxel | 0.23 | 0.021 | <0.001 | 60 |
| Actinomycin | 0.172 | 0.015 | <0.001 | 60 |
| Paclitaxel | 0.115 | 0.011 | <0.001 | 60 |
| Rapamycin | 0.065 | 0.007 | <0.001 | 60 |
| Everolimus | 0.037 | 0.005 | <0.001 | 60 |
| Romidepsin | 0.03 | 0.005 | <0.001 | 60 |
| Vincristine | 0.023 | 0.008 | 0.0052 | 60 |
| Dasatinib | 0.022 | 0.005 | <0.001 | 60 |
| SN38 | 0.022 | 0.006 | 0.0002 | 60 |
| Clofarabine | 0.009 | 0.004 | 0.0194 | 60 |
| FluroaraA | 0.008 | 0.005 | 0.1193 | 60 |
| Imatinib | 0.008 | 0.004 | 0.058 | 60 |
| Tamoxifen | 0.006 | 0.004 | 0.0952 | 60 |
| Azacytidine | 0.004 | 0.004 | 0.2717 | 60 |
| Vorinostat | 0.004 | 0.004 | 0.272 | 60 |
| Nilotinib | 0.004 | 0.004 | 0.3531 | 60 |
| Sunitinib | 0.002 | 0.004 | 0.6086 | 60 |
| Tretinoin | 0 | 0.004 | 0.9995 | 60 |
| Capecitabine | −0.001 | 0.004 | 0.7213 | 60 |
| Lapatinib | −0.003 | 0.005 | 0.562 | 60 |
| Nelarabine | −0.003 | 0.004 | 0.376 | 60 |
| Carboplatin | −0.004 | 0.004 | 0.3224 | 60 |
| Temozolomide | −0.005 | 0.004 | 0.2555 | 60 |
| Gemcitabine | −0.007 | 0.005 | 0.1485 | 60 |
| Celecoxib | −0.008 | 0.004 | 0.0305 | 60 |
| Cytarabine | −0.008 | 0.004 | 0.0477 | 60 |
| Thiotepa | −0.014 | 0.005 | 0.0029 | 60 |
| Floxuridine | −0.016 | 0.005 | 0.0012 | 60 |
| Cladribine | −0.018 | 0.004 | <0.001 | 60 |
| Letrozole | −0.018 | 0.004 | <0.001 | 60 |

ESA: synergistic effect size; seESB: standard error of synergistic effect size; pC: statistical significance value; CountD: number of cell lines tested in the panel; Among these 30 chemotherapeutic agents tested, only TAS108 combined with Docetaxel, Actinomycin, Paclitaxel, Rapamycin, Everolimus or Romidepsin demonstrated significant synergy across the cell line panel. We also aggregated the effects from the 6 synergistic combinations across cancer cell lines with different tissue of origin, with each combination demonstrating significant and consistent synergy across the different tissues, particularly melanoma, ovarian, CNS, renal, lung, breast, colon, prostate, and leukemia cancers.

What is claimed is:

1. A method for inhibiting growth of mammalian tumor cells comprising contacting the cells with a synergistic combination of TAS-108 and a synergistic chemotherapeutic drug, wherein the combination more than additively inhibits growth the cells.

2. The method of claim 1 wherein the contacting step comprises administering the combination to a mammal having a tumor comprising the cells.

3. The method of claim 2 wherein one or both of the TAS-108 and drug is administered in an amount subtherapeutic if administered alone.

4. The method of claim 2, wherein one or both of the TAS-108 and drug is administered in an amount less than its IC₅₀ for said cells.

5. The method of claim 2, wherein the TAS-108 is administered at a dosage significantly less than 40 mg.

6. The method of claim 2 wherein the combination is administered in a single composition.

7. The method of claim 2 wherein the drug is an mTOR inhibitor.

8. The method of claim 2 wherein the drug is an mTOR inhibitor that is Rapamycin, Everolimus, Temsirolimus or Ridaforolimus (Deforolimus).

9. The method of claim 2, wherein the drug is a taxane that is Paclitaxel or Docetaxel.

10. The method of claim 2, wherein the drug is Actinomycin.

11. The method of claim 2 wherein the cells are selected from leukemia, renal, melanoma, CNS, lung, ovarian, colon, breast, prostate, and myeloma tumor cells.

12. The method of claim 2 wherein the cells are breast cancer cells.

13. The method of claim 2, wherein the TAS-108:drug ratio is between 10:1 and 1:10.

14. The method of claim 2, wherein the combination exhibits against a plurality of different tissue cancer cell lines selected from breast, leukemia, renal, melanoma, CNS, colon, lung, prostate, and ovarian cancer cell lines, or of breast cancer cell lines selected from BT549, HS578T, MCF7, MDAMB231, MDAMB468 and T47D, greater synergy compared with a composition comprising TAS-108 and an anti-cancer agent that is Vincristine, Dasatinib, SN38, Clofarabine, FluroaraA, Imatinib, Tamoxifen, Azacytidine, Vorinostat, Nilotinib, Sunitinib, Tretinoin, Capecitabine, Lapatinib, Nelarabine, Carboplatin, Temozolomide, Gemcitabine, Celecoxib, Cytarabine, Thiotepa, Floxuridine, Cladribine or Letrozole.

15. The method of claim 2, wherein the method further comprises the step of detecting a resultant inhibition of growth of the cells.

16. A pharmaceutical composition comprising a synergistic combination, with respect to inhibiting growth of mammalian tumor cells, of TAS-108 and a synergistic chemotherapeutic drug.

17. The composition of claim 16 wherein the TAS-108 and drug are coformulated in unit dosage form.
